(19) United States
(12) Patent Application Publication
(10) Pub. No.: US 2020/0121476 A1
(43) Pub. Date: Apr. 23, 2020

(54) TRIAL NECK

(71) Applicant: DEPUY IRELAND UNLIMITED COMPANY, COUNTY CORK (IE)

(72) Inventors: PHILIPPA WITHERS, LONDON (GB); DUNCAN YOUNG, MELBOURN (GB)

(21) Appl. No.: 16/500,862

(22) PCT Filed: Apr. 9, 2018
(86) PCT No.: PCT/EP2018/059063

§ 371 Date: Oct. 4, 2019

(30) Foreign Application Priority Data
Apr. 12, 2017 (GB) 1705919.7

(51) Int. Cl. A61F 2/46; A61F 2/36; A61B 17/16

(52) U.S. Cl. CPC A61F 2/4684; A61F 2/3609; A61F 2002/30538; A61B 17/1659; A61B 17/1668; A61F 2/4607

(57) ABSTRACT

A bent trial femoral neck (400) and method for visually assessing the orientation of an acetabular cup implanted in a pelvis of a patient are described. The bent trial femoral neck comprises a body (402) extending along a femoral neck axis, an attachment formation (408, 410) extending from the body for releasably attaching the trial femoral neck to a femoral part received with a femur of the patient and a taper (404). The taper extends along a taper axis and the taper axis is tilted anteriorly relative to the femoral neck axis by an angle configured to position a trial femoral head having an indicium on an outer surface so that the indicium is parallel to a plane of a rim of the acetabular cup, or a rim of a liner within the acetabular cup, when the acetabular cup is oriented in the pelvis of the patient with a target orientation and the hip joint is in a predetermined position.

The present invention relates to hip surgery and in particular to apparatus and methods which may be used during hip surgery to visually assess acetabular cup orientation.

A variety of methods of hip surgery are generally known. The hip joint may generally be considered a ball and socket joint in which the head of the femur articulates within the acetabular cavity of the pelvis. Some methods of hip surgery may involve the replacement of one or more parts of the hip joint with one or more prosthetic components. This may be to replace damaged, worn, diseased or otherwise imperfect parts of the hip joint including the respective articulating surfaces of the acetabular cavity and/or femoral head.

Different surgical procedures may involve replacement of a part of the acetabulum or the femoral head or both. Some procedures, sometimes referred to as resurfacing procedures, may involve replacement of only the articulating surface of the femoral head. Other procedures may involve replacement of the entire femoral head. Such procedures often also use a femoral or stem component which is implanted in the resected femur and having a neck to which the femoral head is attached. In some procedures, a prosthetic cup may be implanted in a prepared acetabular cavity to provide a cavity in which the femoral head or prosthetic femoral head may articulate when the joint is reduced. Surgical procedures in which both the acetabulum and at least a part of the femoral head are replaced with prosthetic components are generally referred to as total hip replacement procedures.

During hip surgery procedures, some surgeons may sometimes use one or more trial components, which have the same geometry and size as the intended prosthetic components, so that the surgeon may trial the joint before final implantation of one or both of the prosthetic components. For example, the surgeon may use trial components to check that the size, position or orientation of one or more of the components is suitable. Other surgeons may opt not to use trial components or may use them occasionally based on their professional judgement.

One of the considerations in hip surgery is the angular orientation of the acetabular cavity. When the acetabulum is replaced with an acetabular cup, then it is often an aim of the surgeon to place the acetabular cup so that it is pointing generally in a preferred direction or range of directions. The orientation of an acetabular cup is often defined in terms of an angle of abduction, or inclination, and an angle of anteversion.

A variety of approaches have been used to try and assess the angular orientation of an acetabular cup, either a trial or a prosthesis, after placement in the acetabular cavity.

Anatomical approaches may be used in which the surgeon uses either their experience and/or a piece of instrumentation, in order to gauge, by inspection or instrumentation, the angular orientation of the acetabular cup relative to one or more anatomical features of the patient's pelvis. However, there is often limited access to the surgical site, particularly for minimally invasive approaches, and therefore this is often not easy nor accurate.

Other approaches may use markings or other features on the trial or prosthetic components in order to gauge the angular orientation of the acetabular cup relative to the patient's pelvis.

For example “The Ranawat Sign A Specific Maneuver to Assess Component Positioning in Total Hip Arthroplasty”, Lucas, David H., and Scott, Richard D., Journal of Orthopaedic Techniques, Vol. 2, No. 2, June 1994, describes a method of intraoperative assessment of component orientation for total hip arthroplasty. With the patient in the true lateral decubitus position, the femur is internally rotated without hip flexion until a flat underside of the prosthetic head (generally perpendicular to the femoral neck) is co-planar with a rim of the acetabular cup. The amount of internal rotation necessary to achieve this position is known as the Ranawat sign and relates to the combined anteversion of the acetabular and femoral components of the joint. For example a Ranawat sign of 45° may correspond to a cup anteversion of 30° and a femoral anteversion of 15°. However, any knee laxity or deformity can influence the interpretation of this value. Also, there may be difficulty in assessing the actual magnitude of the angle of internal rotation. Further, different surgeons may have different approaches to manipulating the patient's leg and also any one surgeon's approach may not easily be reproducible from patient-to-patient either by that same surgeon or by other surgeons.

WO 2009/108683 describes another approach in which markings are applied to a femoral head and in which the surgeon again applies an amount of internal rotation to the patient's leg, during trial reduction, so that the angle between the rim of the acetabular cup and various markings on the femoral head indicates the angular position of the acetabular cup relative to the patient's pelvis. A leg position is used in which the patient's leg is in full, relaxed extension at zero degrees abduction, zero degrees anteversion and approximately ° internal rotation, or otherwise internally rotated by an amount equal to the amount of version of the natural or artificial femoral neck. Hence, this approach also requires the surgeon to apply a specific amount of internal rotation to the patient's leg. Again, it may be difficult to apply the correct amount of internal rotation, there may be inaccuracies introduced by deformities of the patient's leg and the surgical technique may be difficult to reproduce and/or reliably learn. This is particularly the case for a relatively small angle, 15°, as even a relatively small error in the amount of internal rotation, for example 5°, is a large proportion (33%) of the target internal rotation.

Other approaches and associated instrumentation are described in US 2005/0107799. An accessory for implanting a hip cup, includes a manipulable cup, a manipulation head having a hemispherical portion and a circular rim around it for aligning the manipulable cup in the acetabulum. A device for immobilizing the aligned position of the manipulable cup is provided and allows a guide to be set for alignment of a bone bur and a drive-in instrument for reaming placing the acetabular cavity and placing the cup. In another approach, a manipulable cup is located in the acetabulum and its orientation can be adjusted by a handle until a lip of the manipulable cup is parallel with an equatorial line on a femoral head or a plane on the reverse of a femoral head Hence, apparatus and/or methods making accurate intraoperative assessment of acetabular cup placement simpler, easier and/or more reliable would be beneficial.

A first aspect of the invention provides a bent trial femoral neck for visually assessing the orientation of an acetabular cup implanted in a pelvis of a patient, the bent trial femoral neck comprising: a body extending along a femoral neck axis; an attachment formation extending from the body for releasably attaching the trial femoral neck to a femoral part received with a femur of the patient; and a taper, wherein the taper extends along a taper axis and wherein the taper axis is tilted relative to the femoral neck axis by an angle configured to position a trial femoral head having an indicium on an outer surface so that the indicium is parallel to a plane of a rim of the acetabular cup, or a rim of a liner within the acetabular cup, when the acetabular cup is oriented in the pelvis of the patient with a target orientation and the hip joint is in a predetermined position.

The taper may be tilted out of the plane defined by a central plane of the body and in which the neck axis lies.

The taper axis may be tilted anteriorly relative to the femoral neck axis. The taper may be tilted in a generally anterior direction relative to the body or the neck axis. The taper may be tilted relative to the neck about an axis generally parallel to the inferior-superior axis.

The taper axis may be tilted relative to the femoral neck axis by an angle in the range of 10° to 40°.

The taper axis may be tilted relative to the femoral neck axis by an angle of 35°.

The taper axis may be tilted relative to the femoral neck axis by an angle of 20°.

The body may comprise a lower and an upper portion and wherein a recess is located on a side of the bent trial femoral neck between the upper portion and the lower portion and arranged to accommodate a portion of the trial femoral head when attached to the taper.

30 The lower portion and the upper portion may be connected by a rib located on a side of the bent trial femoral neck opposite to the recess.

The target orientation may be a target anteversion of the acetabular cup.

The target anteversion of the acetabular cup may be 20° relative to the pelvis of the patient.

The hip joint may be in an anatomical position corresponding to the femur of the patient being placed in 0° of flexion/extension, 0° of adduction/abduction and 0° of internal/external rotation.

A second aspect of the invention provides a kit of parts comprising: the bent trial femoral neck of the first aspect of the invention; and a trial femoral head, wherein the trial femoral head includes a cavity configured to receive the taper therein and wherein the cavity extends along a trial head axis and wherein the indicium is arranged to identify a plane perpendicular to the trial head axis.

The indicium may be an equatorial indicium.

The kit of parts may further comprise: a femoral cutting instrument including a further attachment formation and wherein the trial femoral neck is releasably attachable to the femoral cutting instrument using the attachment formation and the further attachment formation.

The femoral cutting instrument may be a femoral broach.

A third aspect of the invention provides an assembly of the kit of parts of the second 25 aspect of the invention, wherein the trial femoral head is mounted on the taper of the bent trial femoral neck.

The trial femoral neck may be attached to the femoral broach.

A fourth aspect of the invention provides a method of visually assessing the orientation of an acetabular cup implanted in a pelvis of a patient, comprising: attaching a bent trial femoral neck to a femoral cutting instrument within a femur of the patient, wherein the bent trial femoral neck has a taper and a body and wherein the taper is tilted relative to the body; attaching a trial femoral head to the taper, wherein the trial femoral head includes an indicium on an outer surface of the trial femoral head; reducing a trial hip joint by locating the trial femoral head within the acetabular cup; positioning the femur of the patient to put the trial hip joint in a predetermined position; and visually inspecting the angle between the indicium and a rim of the acetabular cup or a rim of a liner within the acetabular cup to assess how close the orientation of the acetabular cup is to a target orientation of the acetabular cup.

The orientation may be the anteversion of the acetabular cup.

The anteversion of the acetabular cup may be 20° relative to the pelvis of the patient.

The preselected position may be an anatomical position corresponding to the femur of the patient being placed in 0° of flexion/extension, 0° of adduction/abduction and 0° of internal/external rotation.

The femoral cutting instrument may be a broach or a rasp.

The femoral cutting instrument may be anteverted by 15° relative to the femur of the patient. More specifically, the femoral cutting instrument may be anteverted by 15° relative to the epicondylar axis of the femur.

An embodiment of the invention will now be described in detail, by way of example only, and with reference to the accompanying drawings, in which:

FIG. 1 shows a coronal view of a femur;

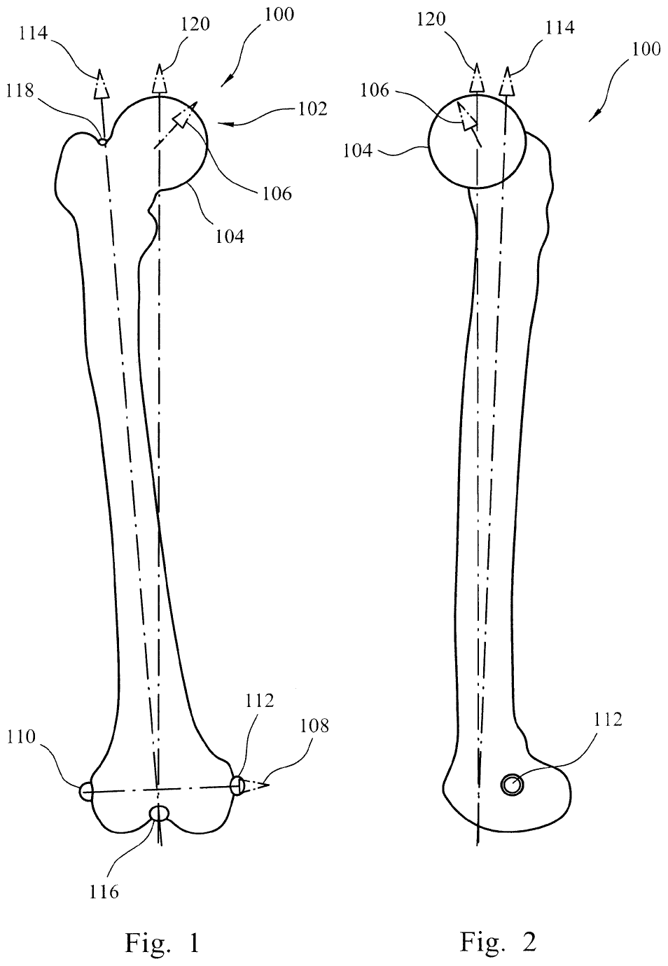

FIG. 2 shows a sagittal view of the femur of FIG. 1;

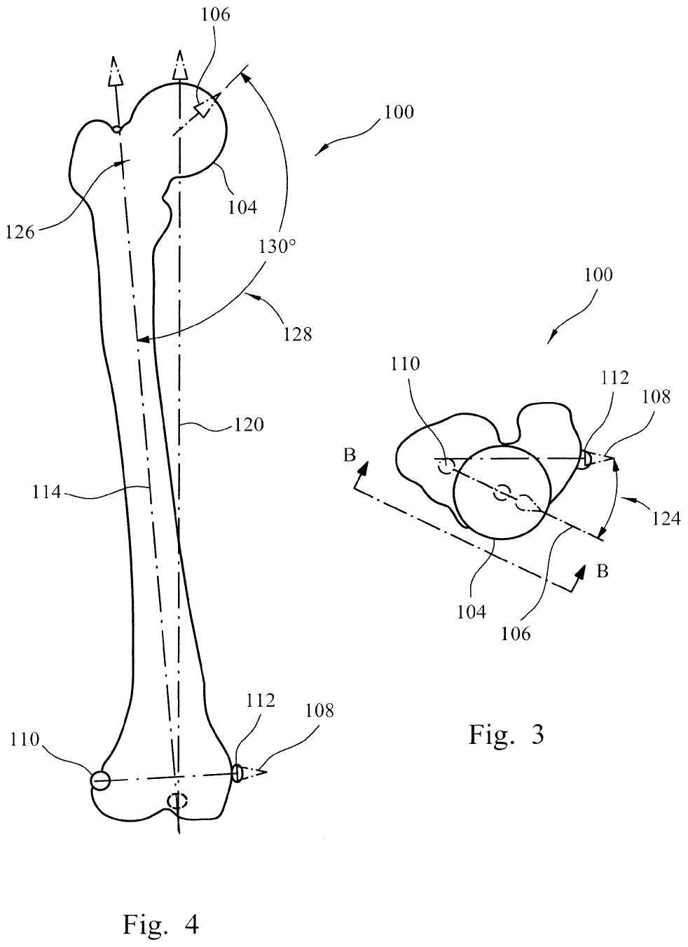

FIG. 3 shows a transverse view of the femur of FIGS. 1 and 2;

FIG. 4 shows a view of the femur in a plane parallel to the anatomic axis and the neck of the femur;

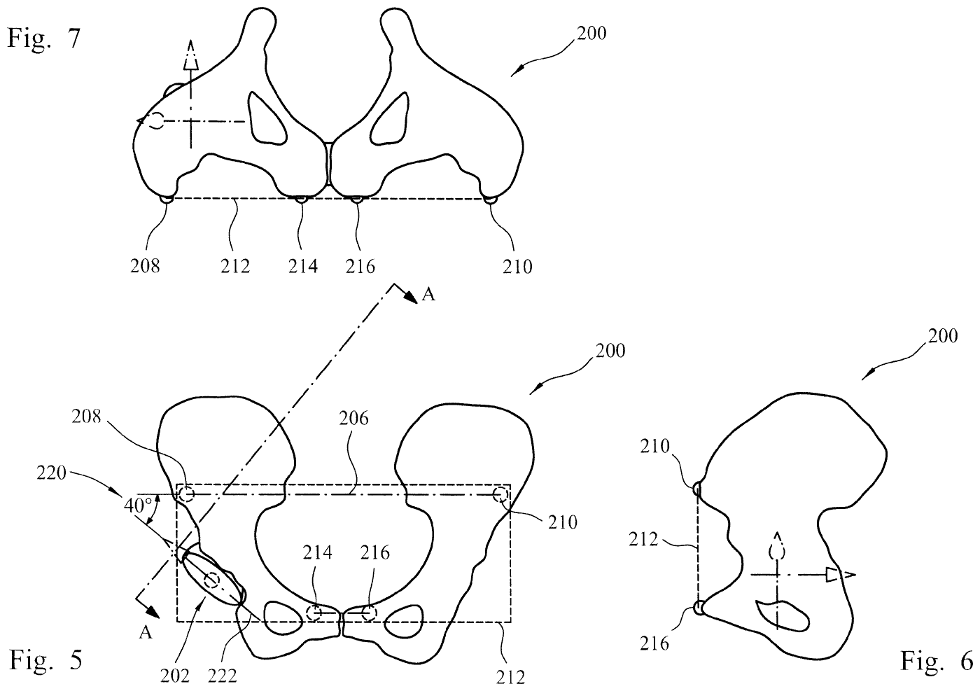

FIG. 5 shows a coronal view of a pelvis;

FIG. 6 shows a sagittal view of the pelvis of FIG. 5;

FIG. 7 shows a transverse view of the pelvis of FIGS. 5 and 6;

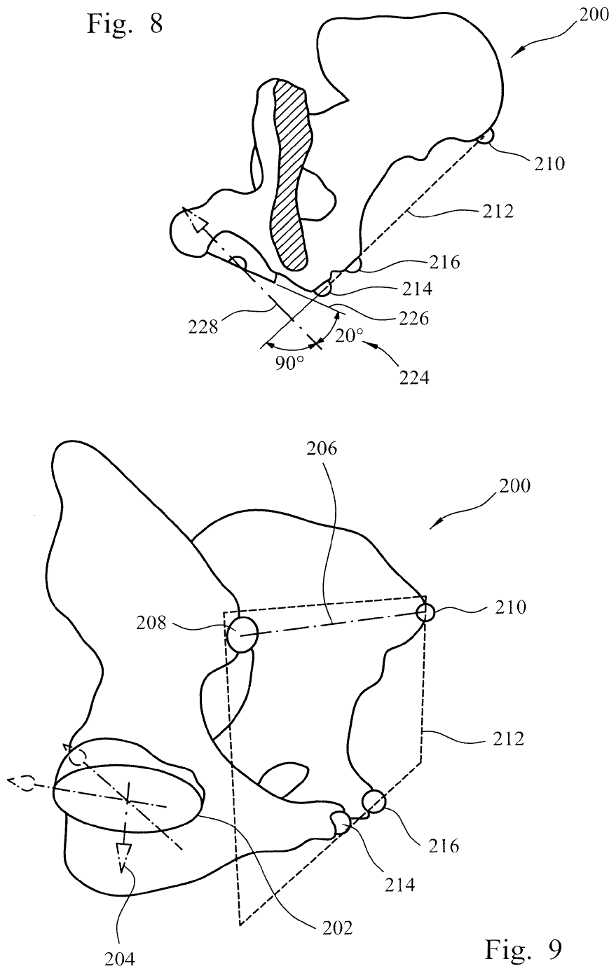

FIG. 8 shows a partial cross sectional perspective view of the pelvis along line A-A of FIG. 5;

FIG. 9 shows a perspective view of the pelvis;

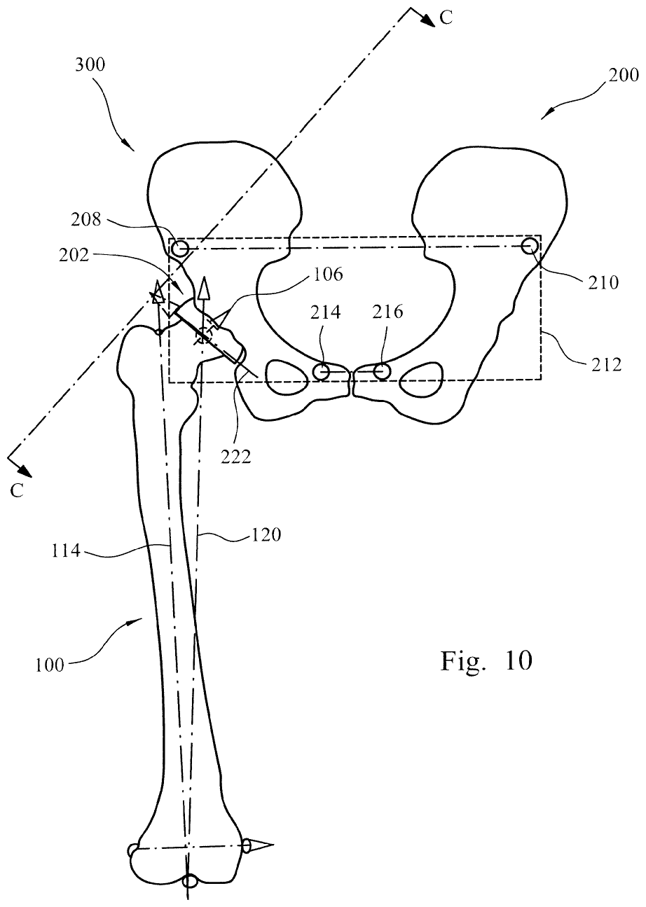

FIG. 10 shows a coronal view of a hip comprising the femur of FIGS. 1 to 4 and the pelvis of FIGS. 5 to 9;

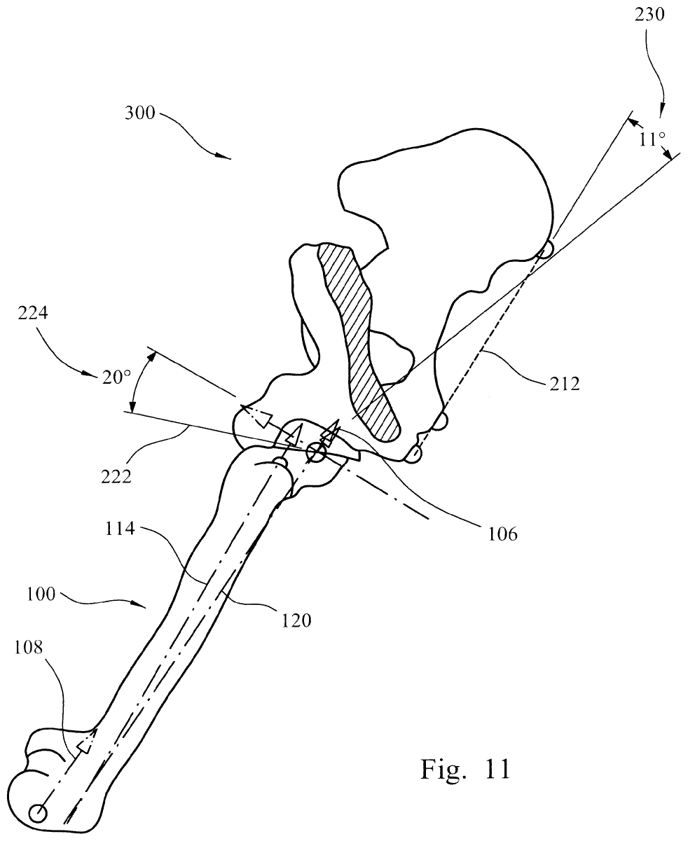

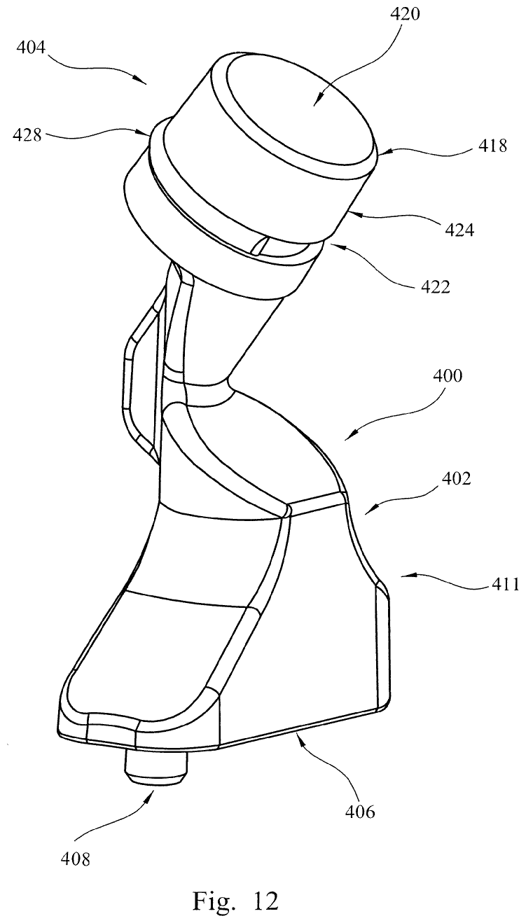

FIG. 11 shows a partial cross sectional perspective view of the pelvis along line C-C of FIG. 10;

FIG. 12 show a perspective view of a first embodiment of a trial neck according to the invention;

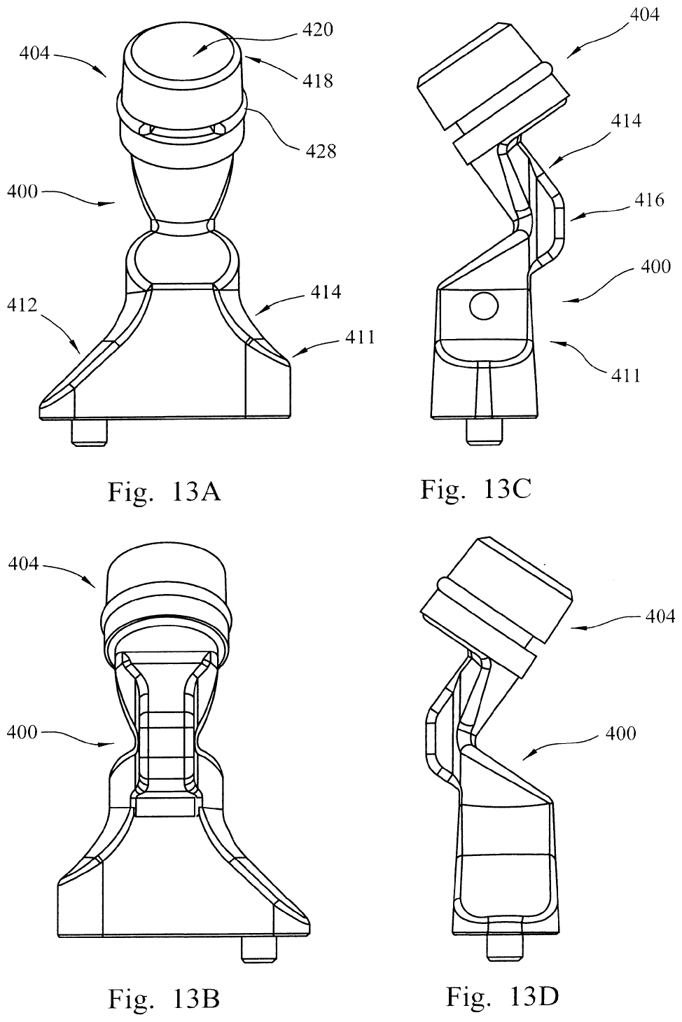

FIGS. 13A to 13D show various views of the trial neck shown in FIG. 12;

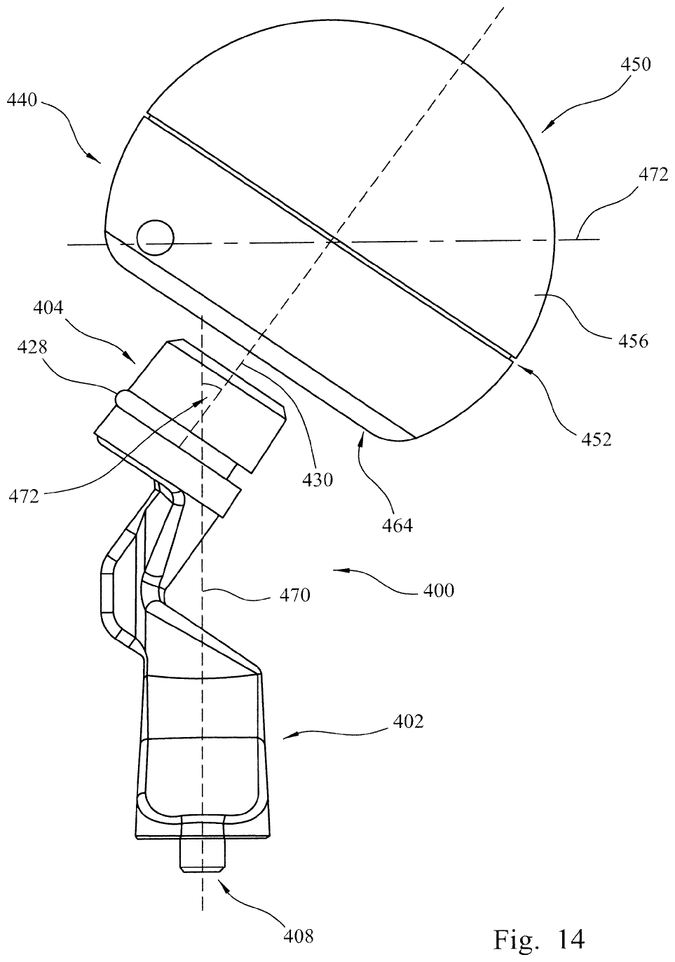

FIG. 14 shows an exploded view of the trial neck shown in FIG. 12 and a trial femoral head which can be assembled into an assembly according to the invention;

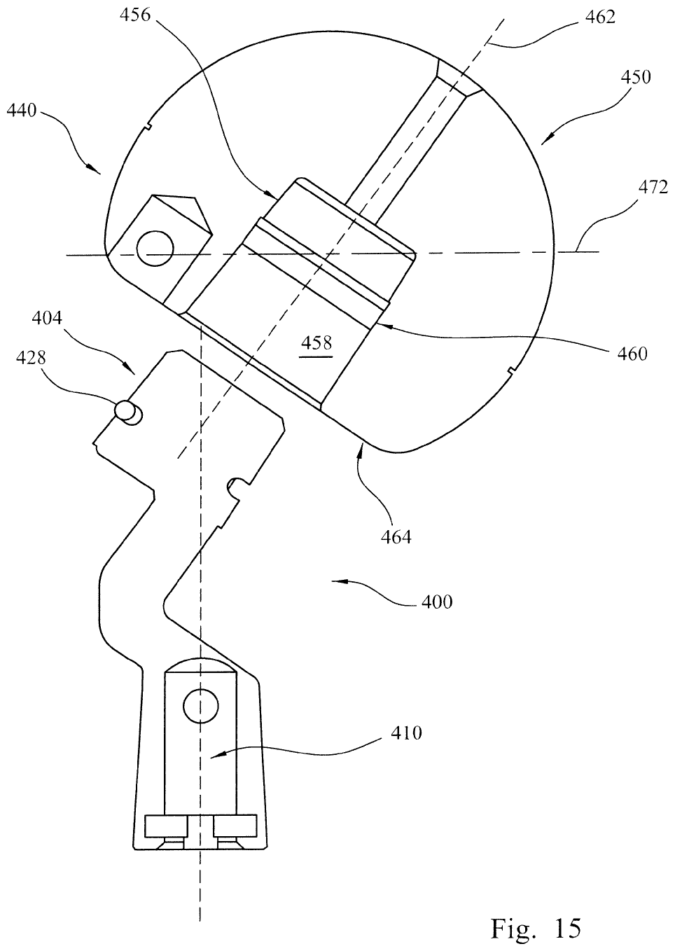

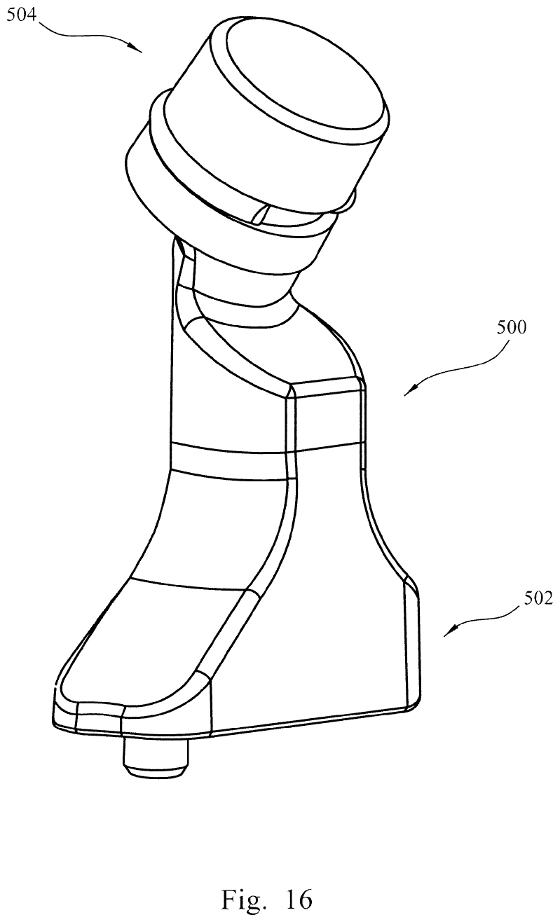

FIG. 15 shows a cross sectional view of the trial neck and trial femoral head shown in FIG. 14;

FIG. 16 shows show a perspective view of a second embodiment of a trial neck according to the invention;

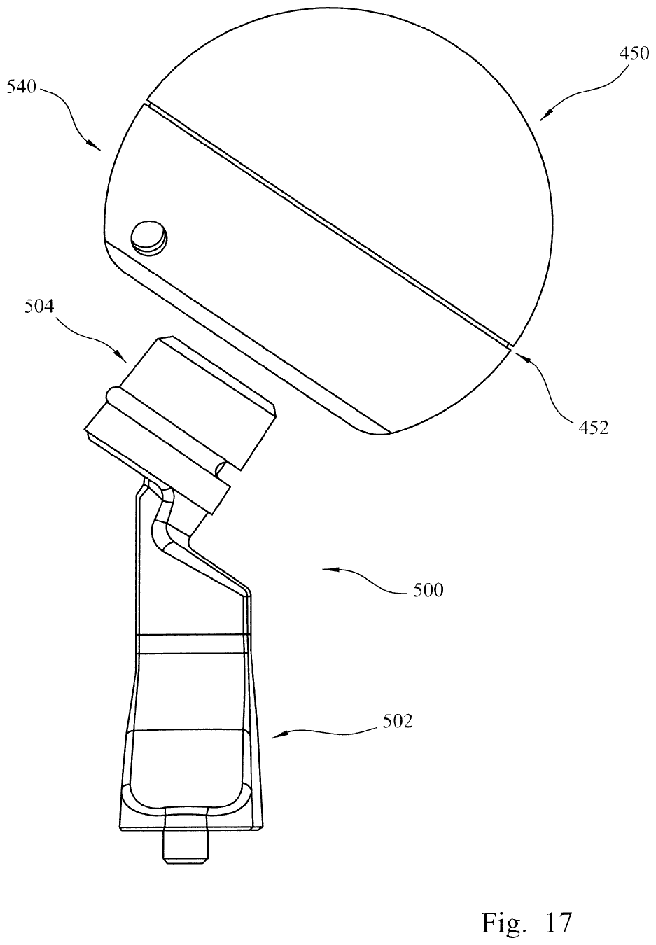

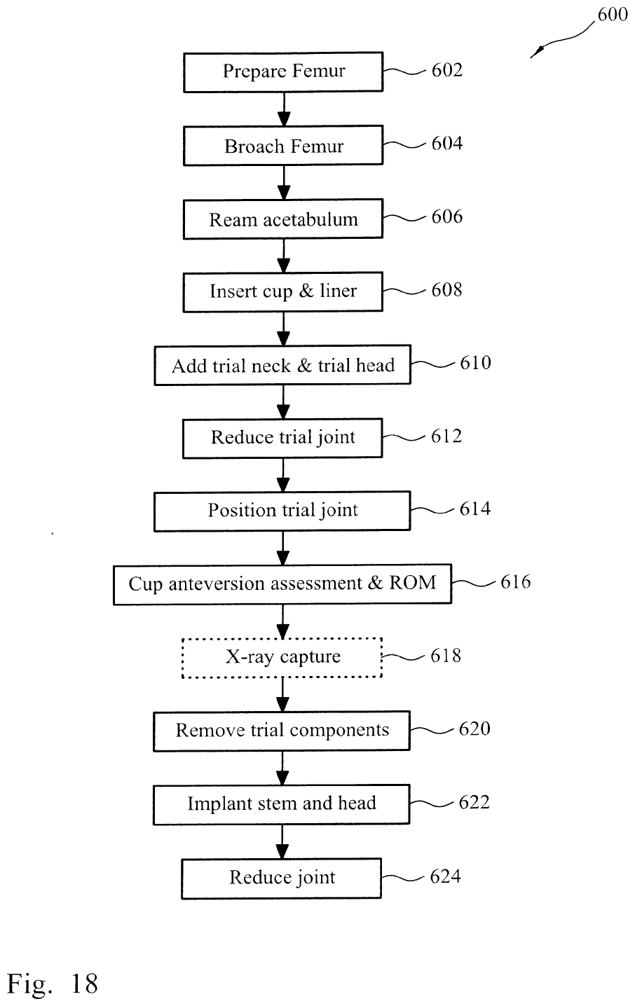

FIG. 17 shows an exploded view of the trial neck shown in FIG. 16 and a trial femoral head which can be assembled into an assembly according to the invention; and

FIG. 18 shows a flow chart illustrating a method of use of the trial neck during a hip replacement surgical procedure according to the invention.

Similar items in different Figure shared common reference signs unless indicated otherwise.

Before describing the apparatus and/or methods of the invention, the geometry of a hip joint will be discussed generally. In the below, a right hip joint is described, but it will be appreciated that a similar discussion applies to a left hip joint. Also, the following discussion is intended to relate to both the pre-operative natural, or native, hip joint, as well as to the artificial, or prosthetic, hip joint. Hence, although the magnitude of the various angles may vary between the native hip joint and the prosthetic hip joint, the definitions of those angles may be generally the same for the native and prosthetic hip and may be determined by the positions and/or orientations of the various parts making up the native hip joint and prosthetic hip joint respectively.

With reference to FIGS. 1 to 4, there are shown various different views of a right femur 100. In particular FIG. 1 shows a coronal view in the anterior to the posterior direction (generally herein the AP direction), FIG. 2 shows a sagittal view in the medial to the lateral direction (generally herein the ML direction), FIG. 3 shows a transverse view in the superior to the inferior direction, and FIG. 4 shows a view of the femur in a plane parallel to the anatomic axis of the femur and the neck of the femur as explained in greater detail below. Pre-operatively, the proximal part of the femur 100 includes the native femoral neck and native femoral head. Intra-operatively and post-operatively the proximal part of the femur may include various trial or prosthetic parts or components providing trial or prosthetic femoral necks and femoral heads. The following will refer generally to femoral necks and femoral heads and is intended to include native, trial or prosthetic ones.

With particular reference to FIGS. 1 and 2, the proximal part 102 of femur 100 includes a femoral head 104 (represented by a sphere) attached to a femoral neck 126, best illustrated in FIG. 4, having a neck axis extending generally in the direction of arrow 106. The femur 100 has an epicondylar axis 108 extending between the lateral femoral epicondyle 110 and the medial femoral epicondyle 112. The femur 100 also has an anatomic axis 114 extending between, for example, the distal femur intercondylar notch 116 and the piriformis fossa, close to the medial face of the greater trochanter. The femur 100 also has a mechanical axis 120 extending between, for example, close to the distal femur intercondylar notch 116 and the centre of the femoral head 104. The anatomical axis 114 and mechanical axis 120 of the femur 100 may be defined by other anatomical points in other embodiments.

With reference to FIG. 3, a femoral neck anteversion angle 124 can be defined as the angle in the transverse plane subtended by the femoral neck axis 106 and the epicondylar axis 108. In practice, the femoral neck anteversion angle for the native neck is typically in the range of about 12° to 15°, but may have other values. Neck anteversion angle 124 is a measure of the anteversion of the femoral neck relative to the local anatomy of the femur 100.

FIG. 4 shows a view of the femur 100 in a plane parallel to line BB of FIG. 3, which is parallel to the femoral neck axis 106, and the anatomical axis 114, and which more clearly shows the femoral neck 126. FIG. 4 also illustrates the neck angle 128 subtended between the femoral neck axis 106 and the anatomical axis 114 of the femur. The native neck angle 128 varies from patient to patient, but is typically about 130°. The neck angle 128 for a trial or prosthetic implant is usually fixed by the implant design, unless the implant is adjustable, and is often intended to approximately reproduce the native geometry and so may also be about 130°. In the following a neck angle 128 of 130° may be used as an example, but it will be appreciated that in other embodiments, other neck angle values may also be used.

Hence during hip surgery in which a prosthetic femoral component is used, one of the variables is the femoral neck anteversion angle 124, which generally measures how far forward the femoral neck 126 is directed compared to the medial-lateral axis of the femur.

With reference to FIGS. 5 to 9, there are shown various different views of a pelvis 200 and right acetabulum. In particular FIG. 5 shows a coronal view in the anterior to the posterior direction (generally herein the AP direction), FIG. 6 shows a sagittal view in the medial to the lateral direction (generally herein the ML direction), FIG. 7 shows a transverse view in the superior to the inferior direction, FIG. 8 shows a partial sectional view along line A-A of FIG. 5, and FIG. 9 shows a perspective view of the pelvis 200 with the anterior pelvic plane (APP) vertical and the transverse axis generally horizontal. Pre-operatively, the pelvis 200 includes an acetabulum which provides a native socket in which the native femoral head is received and articulates. Intra-operatively and/or post-operatively the pelvis may include various trial or prosthetic implants, such as trial or prosthetic acetabular cups (with or without liners depending on the specific implant system being used). The following will refer generally to the acetabulum or acetabular cup and is intended to include the native acetabulum as well as trial or prosthetic components.

As illustrated in FIG. 5, the acetabulum 202 may be represented by a hemisphere or hemispherical cup which generally has a position and an orientation. The orientation or direction of the acetabulum may generally be defined by two angles. A first angle indicates how much the acetabulum is directed forward or backward (generally referred to as anteversion when pointing anteriorly and retroversion when pointed posteriorly) relative to the pelvis. A second angle indicates how much the acetabulum is pointing downward or in an inferior direction (generally referred to as inclination or abduction) relative to the pelvis. The direction of the acetabulum may be defined by an acetabular axis 204, best illustrated in FIG. 9, which generally passes through the centre of the mouth of the acetabulum and perpendicular to the plane of the mouth of the acetabulum.

The pelvis 200 includes a transverse axis 206 passing between the right ASIS 208 and the left ASIS 210. An anterior pelvic plane 212 (generally referred to as APP in the following) is defined by the transverse axis 206 and first and second points on the symphysis pubis 214, 216.

As best illustrated in FIG. 5, an inclination angle for the acetabulum or acetabular cup 202 may be defined by the angle 220 subtended by the transverse axis 206 and a long axis, or inclination axis, 222 of the acetabular cup 202 within, or parallel to, the anterior pelvic plane 212. In FIG. 5, the illustrated inclination angle 220 is approximately 40°. FIG. 8 shows a view of a cross section of the pelvis 200 along line A-A in FIG. 5 and in a direction along the long axis 222 of the acetabular cup 202. Hence, FIG. 8 shows the plane generally perpendicular to the long axis 222 of the pelvic cup 202. From FIG. 8, an anteversion angle 224 may be defined as the angle subtended between the plane 226 of the mouth of the acetabular cup and a plane 228 perpendicular to the anterior acetabular plane 212. Hence, as illustrated in FIG. 8, the acetabulum or acetabular cup 202 has an anteversion angle 224 of approximately 20°.

Hence, as illustrated in the perspective view of the pelvis 200 in FIG. 9, the acetabulum or acetabular cup 202 has an orientation corresponding to an inclination of 40° and an anteversion of 20°. These angles may be referred to as radiographic angles as they are based on the APP view of the pelvis illustrated in FIG. 5 and which is the view of the pelvis typically radiographically imaged or X-rayed and which images are often used by surgeons pre-, intra- and/or post operatively to assess acetabular orientation.

With reference to FIGS. 10 to 11, there are shown various different views of a right hip joint 300 formed by femur 100 and pelvis 200. In particular, FIG. 10 shows a coronal view in the anterior to the posterior direction (generally herein the AP direction), similar to FIGS. 1 and 5 combined. In FIG. 10, the femur has been placed in an anatomical position as described in greater detail below. FIG. 11 shows a partial section along line C-C of FIG. 10 and viewed in a direction along the inclination axis 222 of the acetabular cup 202. Hence, the plane of FIG. 11 is generally perpendicular to the direction of the inclination axis 222 of the acetabular cup.

As illustrated in FIG. 11, the acetabular cup 202 has an anteversion angle 224 of 20° in the plane perpendicular to the inclination axis 222 of the acetabulum. FIG. 11 also illustrates the femoral anteversion angle 230 in the plane perpendicular to the inclination axis of the acetabulum and being defined by the angle 230 subtended in that plane by the femoral neck axis 106 and the anterior pelvic plane 212. As illustrated in FIG. 11, the apparent femoral anteversion angle 230 is approximately 11°.

Hence, as can be seen the overall geometry of the hip joint arises from the orientation of the acetabulum relative to the pelvis and also the orientation of the femoral neck relative to the femur. In the illustrated example, the overall or combined anteversion of the hip joint 200 shown in FIGS. 10 and 11 is the combination of the acetabular anteversion, about 20°, and the amount of femoral anteversion projected into the same plane, which in this example is approximately 11°. Hence, the combined anteversion of the hip joint 300 in this plane is about 31°.

However, in practice, when surgeons talk about a combined anteversion of about 35°, this may be arrived at by adding absolute values of angles in different planes, 15° anteversion of the neck relative to the femur in a first plane and 20° anteversion of the acetabulum relative to the pelvis in a second, different plane, to give approximately 35°. In practice, the combined anteversion is assumed to be in the range of approximately 30° to 40°, as being typically greater than 30° and less than 40°, and that any measurement or assessment is likely to be accurate to plus or minus a few degrees anyway and so measurement of the angles in different, non-parallel planes is not crucial.

Herein, combined anteversion may refer, depending on the context, to the general idea that the anteversion of a hip joint is the combined effect of the degree of anteversion of the femoral neck relative to the femur and also the degree of anteversion of the acetabulum relative to the pelvis. More specifically, for non-extreme cases, combined anteversion may also refer to a general rule of thumb that the sum of the acetabular anteversion and the femoral anteversion, measured in the same plane, should have a certain value, for example approximately 35°. Hence, if a low value of one occurs, then the other can be increased (or vice versa) in order to bring the combined anteversion closer to this target value.

With reference to FIG. 12 there is shown a perspective view of a first embodiment of a trial femoral neck 400 according to the invention. FIG. 12 shows a trial femoral neck for a left hip. A trial femoral neck for a right hip is generally similar but is a mirror image of the right hip trial femoral neck 400. As can be seen in FIG. 12, the trial neck 200 has a generally bent shape or form and hence provides a bent trial femoral neck.

FIG. 13A shows a view of a first side of the trial femoral neck 400, corresponding generally to a medial-lateral direction in use, FIG. 13B shows a view of a second side of the trial femoral neck, corresponding generally to a lateral-medial direction in use, FIG. 13C shows a view of first end of the trial femoral neck 400, corresponding generally to a superior-inferior direction in use and FIG. 13D shows a view of a second end of the trial femoral neck 400, corresponding generally to an inferior-superior direction when in use.

The trial femoral neck 400 comprises a body 402 and a taper 404 at a free end of the trial femoral neck and which is tilted or inclined relative to the body 402. The taper 404 is retroverted relative to the remainder of the trial femoral neck. The body has an underside 406 from which a first attachment formation 408, in the form of a circular peg, extends. Ad best illustrated in the cross sectional view of FIG. 15, the body 402 also defines a cavity 410 therein, which provides a second attachment formation. A lower portion 411 of the body 402 has a first shoulder 412 and a second shoulder 414. An upper portion 414 of the body 402 is connected to the lower portion by a rib 416. The taper 404 is provided at a free end of the upper portion 414. The taper 404 has a chamfer 418 running around the periphery of its end face 420. A shallow groove 422 is formed in and extends around a side wall 424 of the taper. A partial ring or C-clip 428 of resilient material, such as stainless steel, nylon or acetal, is located in the groove and extends around the majority of the taper. The C-clip acts to provide a friction fit with a trial femoral head as described in greater detail below.

The first 408 and second attachment formations 410 are arranged to co-operate with counterpart respective female and male attachment formations provided in a superior portion of a femoral part, such as a femoral broach or rasp, to allow the trial femoral neck 400 to be releasably attached to the formal part in use, as described in greater detail below.

The trial femoral neck 400 may be made from any suitable biocompatible material such as a metal, alloy or plastic. In particular, the trial femoral neck 400 may be made from stainless steel. Alternatively, the trial femoral neck may be made from a plastic, for example a glass-filled PAEK/PEEK or a PAEK/PEEK Blend such as Solvay Avaspire.

Other suitable materials include titanium, aluminium, cobalt chrome, polyphenylsulphone, polyacetal, polyamide, polypropylene, polyarylamide, polyetherimide, acrylonitrile-butadiene-styrene, polymethylmethacrylate, polycarbonate, and all polymers could be unfilled state or filled with glass or carbon fibres or beads.

FIG. 14 shows an exploded view of an assembly 440 also according to the invention and comprising the trial femoral neck 400 and a trial femoral head 450. The trial femoral head 450 is generally in the form of a truncated sphere. The trial femoral head includes an indicium, in the form of a shallow groove 452 in the outer surface 454 of the trial femoral head, and which extends around an equator of the trial femoral head. FIG. 15 shows a cross sectional view of the trial femoral neck 400 and trial femoral head 450 in the same orientation as FIG. 14.

As best illustrated in FIG. 15, the trial femoral head 450 has an inner wall 456 defining a cavity 458 within the trial femoral head 450. The cavity is configured to receive the taper 404 therein and includes a slightly recessed portion 460 extending around the cavity 458 and arranged to interface with the C-ring 428 to provide a friction fit to allow the trial femoral head 450 to be releasably attached to the trial femoral neck while also being securely retained on the trial femoral neck. The trial femoral head cavity 458 extends along a femoral head axis of the trial femoral head, indicated by dashed line 462 and which passes through a pole of the trial femoral head and is generally perpendicular to a flat underside 464 of the trial femoral head. The trial femoral head axis 462 is coincident with a taper axis 430 in FIGS. 14 and 15. The equatorial indicium 452 lies in a plane generally perpendicular to the trial femoral head axis 462.

Although an equatorial indicium is used in the described embodiment, in other embodiments, the indicium may be at other positions along the trial femoral head axis. All that is required is that the indicium generally indicates a plane which is generally perpendicular to the trial femoral head axis. Hence in other embodiments, the indicium may be other lines of constant latitude, which are parallel to the actual equator of the trial femoral head.

Also, although a groove is used as the indicium, other embodiments, the indicium may be provided by other features, such as rib, a line or band on the surface, whether continuous or broken, and similar.

As best illustrated in FIG. 14, the taper 404 extends along a taper axis 430 and the body 402 of the trial neck extends along a femoral neck axis 470. In use, when mounted on a femoral component, the femoral neck axis 470 generally extends along the neck axis 106. A conventional trial neck is generally straight and the taper also extends in the same direction as the body of the neck and along the neck axis. However, in the invention, the taper is tilted or inclined relative to the rest of the trial neck and hence the trial neck may be referred to as a bent trial neck. In particular, the taper 404 is tilted relative to the body 402 and the taper axis 430 subtends an angle 472 of approximately 35° with the femoral neck axis 470. The taper is effectively retroverted by the desired angle of combined anteversion, in this example approximately 35°.

As illustrated in FIG. 14, the taper 404 is tilted in a generally anterior direction relative to the body 402 or the neck axis 470. FIG. 12 shows a view of the plane generally defined by the medial-lateral and anterior-posterior axes. Hence, the taper 404 is tilted relative to the neck about an axis generally parallel to the inferior-superior axis. Put another way, the taper is tilted out of the plane defined by the central plane of the body 402 and in which the neck axis 470 lies.

The taper 404 is configured such that when the trial head 450 is fully seated on the taper, then the centre of the trial head 450, corresponds to the same position, that the trial head 450 would have if used with a conventional trial neck of the same length. Hence, the position of the centre of rotation provided by the trial head 450 when mounted on the trial neck 402 is the same, but the orientation of the trial head is tilted by an angle of approximately 35° relative to broach or neck of the stem. Hence, the equatorial indicium is also tilted away from the ‘horizontal’ in FIGS. 14 and 15, as represented by chain-line 474. The angle of inclination of the taper 472, is selected so that when the assembly 440 of the trial neck and trial head is mounted on an implanted femoral component, and the joint reduced and placed in a predetermined position, then the equatorial indicium 452 will be parallel to the rim of an acetabular cup, or liner, which has been placed with a preferred or target anteversion angle and preferred or target inclination angle in the pelvis of the patient.

Specifically, the angle of inclination 472 corresponds to a target cup anteversion of approximately 20° relative to the patient's pelvis when the assembly 440 is approximately 15° anteverted relative to the patient's femur and the trial hip joint is placed in an anatomical position or configuration. The anatomical position of the hip joint corresponds to the patient's femur being placed in 0° of flexion/extension, 0° of adduction/abduction and 0° of internal/external rotation. Hence, the combined anteversion of the hip joint would be approximately 35°, if the rim of the cup or liner is generally parallel to the equatorial indicium 452. The plane of the rim of the cup or liner will be parallel to the plane of the equatorial indicium 452 if the cup has also been placed with an inclination of approximately 40° relative to the pelvis.

FIG. 16 shows a perspective view of a second embodiment of a trial femoral neck 500 according to the invention, and FIG. 17 shows an exploded view in the generally medial-lateral direction of a trial assembly 540, including the trial femoral neck 500 and the trial femoral head 450 with an equatorial indicium. Similarly to the first embodiment, trial femoral neck 500 has a body 502 extending along a femoral neck axis and a taper 504 at a free end of the trial femoral neck and which extends along a taper axis. The taper 504 is tilted or inclined relative to the body 504 and hence trial femoral neck 500 had a generally bent shape or form.

Similarly to the first embodiment as illustrated in FIG. 14, the taper 504 is tilted in a generally anterior direction relative to the body 502 or the neck axis 470. FIG. 17 shows a view of the plane generally defined by the medial-lateral and anterior-posterior axes. Hence, the taper 504 is tilted relative to the neck about an axis generally parallel to the inferior-superior axis. Put another way, the taper is tilted out of the plane defined by the central plane of the body 502 and in which the neck axis lies.

The trial femoral neck 500 is closer to a representation of a “normal” neck and therefore represents more accurately the external envelope of a real implant neck. Hence, during a range of motion (ROM) assessment, the outcomes and assessment would be more representative of a real implant, for example, the neck would clash with the cup at approximately the same angles.

The first embodiment of the trial femoral neck allows greater compatibility but has material absent on one side and added on the other side and so may not as accurately reproduce a ROM assessment for a prosthetic neck.

The trial femoral neck 500 may be made from any suitable biocompatible material such as a metal, alloy or plastic as described above in relation to the first embodiment.

With reference to FIG. 18, there is shown a flow chart illustrating a hip replacement surgical procedure 600 in which the trial femoral necks 400, 500 and assemblies 450, 550, may be used. The order of some of the steps is not relevant but the order of some of the steps may be relevant as will become apparent from the following description. Also, some of the described steps may be optional, and may be omitted, depending on the workflow that a surgeon may prefer.

At 602, the femur is prepared in a generally conventional manner which may include resecting the native femoral neck and head and then at 604 a cavity is formed along the intramedullary canal to accept a femoral stem component and which typically includes using one or more broaches and/or rasps. When a final sized broach has been used, then a broach handle is removed and the broach is left in the femur. At 606, the acetabulum is prepared in a generally conventional manner which may include removing soft tissue and forming a hemispherical cavity within the native acetabulum using an acetabular reamer. At 608 a prosthetic acetabular cup is inserted in the acetabular cavity by the surgeon using a cup inserter and with a certain orientation (version and abduction). Any cup liner may also be inserted in the implanted acetabular cup at 608.

At 610, the bent trial femoral neck 400, 500 is attached to the broach in the femur and the trial femoral head 450 is attached to the taper 402, 502 of the trial neck 400, 500 to form a trial femoral assembly. At 612, the trial joint is reduced by introducing the trial femoral head 450 into the implanted acetabular cup, or liner. Then at 614, the patient's pelvis and/or leg are manipulated to place the trial joint in the preselected position or configuration.

In particular, the patient's leg is placed with the femur in an anatomical position with respect to the pelvis. Specifically, the femur is placed in 0° of flexion/extension, 0° of adduction/abduction and 0° of internal/external rotation.

Assuming that the pelvis on the operating table has taken up an approximately neutral amount of pelvic tilt then this can be achieved by comparing the following three factors. The long axis of the leg relative to the longitudinal axis of the patient can be adjusted by pulling lightly on the lower limb to pull the leg into full extension. If an anterior approach is being used, with the patient supine, then the leg will naturally rest in full extension. Zero abduction/adduction of the legs can be achieved by placing both lower legs (tibia from knee to ankle) parallel and almost touching. An internal/external rotation angle of 0° can be checked using the epicondyle axis of the knee relative to the transverse axis of the pelvis. If the pelvis is level (supine) or vertical (in a lateral decubitus approach) on the table, then the epicondyle axis can be compared to the table. If a posterior approach is being used, with the patient lateral, then the tibia can be flexed 90° to provide a more discernible indication on the femoral articular axis which will indicate any internal/external rotation of the femur. Hence, depending on the orientation of the patient on the table, the patient's legs are placed in appropriate positions to provide 0° of flexion/extension, 0° of abduction/adduction and 0° of internal/external rotation of the femur with respect to the pelvis. With the patient's legs in this anatomical position, an assessment of the cup orientation can be carried out at step 616.

If the cup has been placed with an orientation having an anteversion relative to the pelvis corresponding to the value to which the bent trial femoral neck has been configured, e.g. 20°, then the equatorial indicium 452 will be generally parallel to the rim of the cup, or liner. Alternatively, if the cup has been placed with an orientation having an anteversion different to the target value, then the equatorial indicium 452 will be not be parallel to the rim of the cup or liner. Hence, the equatorial indicium 452 provides a visual indication of the anteversion angle of the acetabular cup, as the angle subtended between the line of the equatorial indicium 452 and plane of the rim of the cup, or liner, indicates generally how far away angularly the cup has been placed compared to the target anteversion value. If the cup has also been placed with a target inclination angle relative to the pelvis, and which is 40° in this example, then the plane of the rim of the cup or liner will be parallel to the plane of the equatorial indicium. Hence, at 616 the surgeon may assess the cup anteversion by visually inspecting the trial joint assembly to see how close the equatorial indicium 452 is to parallel to the plane of the mouth of the cup or liner.

Optionally, at step 616 a range of motion (ROM) assessment can also be carried out. This is generally known in the art and involves articulating the trial hip joint to detect the likely range of articulation possible before impingement of the hip components.

At 618 an intra-operative X-ray image of the trial joint in the anterior-posterior direction (corresponding to FIG. 5) may optionally be captured with the trial joint in the preselected position.

It is not necessary that the surgeon carryout any repositioning of the cup. The trial assessment at 616 may be carried out simply to assess the anteversion angle achieved so as to provide some immediate intra-operative feedback of the cup orientation to the surgeon.

At 620, all the trial femoral components, including the broach, are removed from the femur. Then at 622, the actual prosthetic femoral stem is implanted in the femoral cavity and the prosthetic femoral head is attached. Then at 624, the joint can be reduced and any range of motion trial carried out if desired. The surgical procedure then substantially ends.

The bent trial femoral neck 400, 500 provides a number of advantages. The bent trial neck 400, 500 can be used with conventional trial femoral heads with an equatorial indicium rather than using special trial heads having specialised alignment markings. Hence, the bent trial neck 400, 500 may be backward compatible with existing trial heads and liners. Also, multiple bent trial necks may be provided each with a different inclination configured to check a different angle. For example, a direct anterior surgical approach might have a target combined anteversion of 20°, whereas a posterior surgical approach may have a target combined anteversion of 35°. Specific left hip and right hip necks may be provided which can help to prevent any confusion when assembling a trial head on the appropriate bent trial neck for a right or left hip. Also, the trial heads do not need to be attached to the taper 404, 504 with any particular rotational angle making assembly of the trial assembly simpler and easier.

In this specification, example embodiments have been presented as particular combinations of features. However, a person of ordinary skill in the art would understand that many other embodiments may be practiced which include a different combination of features, including fewer features or a greater number of features. It is intended that the following claims cover all possible embodiments.

Any instructions and/or flowchart steps may be carried out in any order, unless a specific order is explicitly stated or would be understood to be required from the context of the description. Also, those skilled in the art will recognize that while example methods have been discussed, a variety of other differing methods are possible based on other combinations and/or orders of method steps, and are to be understood within the context provided by this detailed description.

While the inventions are amenable to various modifications and alternative forms, specific embodiments are shown by way of example in the drawings and described in detail. It should be understood, however, that other embodiments, beyond the specific embodiments described, are possible as well. All modifications, equivalents, and alternative embodiments falling within the scope of the appended claims are covered as well.

1. A bent trial femoral neck for visually assessing the orientation of an acetabular cup implanted in a pelvis of a patient, the bent trial femoral neck comprising:
a body extending along a femoral neck axis;
an attachment formation extending from the body for releasably attaching the trial femoral neck to a femoral part received with a femur of the patient; and
a taper, wherein the taper extends along a taper axis and wherein the taper axis is tilted anteriorly relative to the femoral neck axis by an angle configured to position a trial femoral head having an indicium on an outer surface so that the indicium is parallel to a plane of a rim of the acetabular cup, or a rim of a liner within the acetabular cup, when the acetabular cup is oriented in the pelvis of the patient with a target orientation and the hip joint is in a predetermined position.

2. The bent trial femoral neck of claim 1, wherein the taper axis is tilted relative to the femoral neck axis by an angle in the range of 10° to 40°.

3. The bent trial femoral neck of claim 2, wherein the taper axis is tilted relative to the femoral neck axis by an angle of 35°.

4. The bent trial femoral neck of claim 1, wherein the body comprises a lower and an upper portion and wherein a recess is located on a side of the bent trial femoral neck between the upper portion and the lower portion and arranged to accommodate a portion of the trial femoral head when attached to the taper.

5. The bent trial femoral neck of claim 4, wherein the lower portion and the upper portion are connected by a rib located on a side of the bent trial femoral neck opposite to the recess.

6. The bent trial femoral neck of claim 1, wherein the target orientation is a target anteversion of the acetabular cup.

7. The bent trial femoral neck of claim 6, wherein the target anteversion of the acetabular cup is 20° relative to the pelvis of the patient.

8. The orientation guide of claim 1, wherein the hip joint is in an anatomical position corresponding to the femur of the patient being placed in 0° of flexion/extension, 0° of adduction/abduction and 0° of internal/external rotation.

9. A kit of parts comprising:
the bent trial femoral neck of claim 1; and
a trial femoral head, wherein the trial femoral head includes a cavity configured to receive the taper therein and wherein the cavity extends along a trial head axis and wherein the indicium is arranged to identify a plane perpendicular to the trial head axis.

10. The kit of parts of claim 9, wherein the indicium is an equatorial indicium.

11. The kit of parts of claim 9, further comprising:
a femoral cutting instrument including a further attachment formation and wherein the trial femoral neck is releasably attachable to the femoral cutting instrument using the attachment formation and the further attachment formation.

12. The kit of parts of claim 11, wherein the femoral cutting instrument is a femoral broach.

13. An assembly of the kit of parts of claim 9, wherein the trial femoral head is mounted on the taper of the bent trial femoral neck.

14. The assembly of claim 13, wherein the trial femoral neck is attached to the femoral broach.

15-20. (canceled)
